(19) United States
(12) Patent Application Publication
(10) Pub. No.: US 2013/0303962 A1
(43) Pub. Date: Nov. 14, 2013

(54) THERAPEUTIC FLUID PREPARATION, DELIVERY, AND VOLUME MANAGEMENT SYSTEMS AND METHODS

(71) Applicant: Christian Bernard, McHenry, IL (US)

(72) Inventor: Christian Bernard, McHenry, IL (US)

(21) Appl. No.: 13/470,569

(22) Filed: May 14, 2012

(51) Int. Cl. A61M 1/36; A61M 1/34; B01D 61/28; B01D 61/32; B01F 15/04; B01F 15/06

(52) U.S. Cl. 604/6.09; 366/141; 366/144; 210/143; 210/646; 210/637

(57) ABSTRACT

A system and method utilizing non-invasive fluid volume or fluid weight monitoring to facilitate mixing of therapeutic fluid components during the preparation of therapeutic fluid for dialysis is described. A therapeutic fluid source is provided in cooperative engagement with a weight measuring device which monitors the fluid volume or fluid weight of the therapeutic fluid in the therapeutic fluid source. Peristaltic pumps are configured to pump therapeutic fluid between a conventional dialyzer and the therapeutic fluid source. The weight measuring device is engaged by a control scheme programmed to operate the pumps. The control scheme includes a feedback loop from the weight measuring device to the pumps to facilitate control of the transmembrane pressure at the dialyzer without measuring TMP. System and methods for priming, ultrafiltration and backflush are also described.

### CROSS-REFERENCE TO RELATED APPLICATIONS

There are no related applications for this application.

### BACKGROUND OF THE INVENTION

1. Field of the Invention

The invention generally relates to systems and methods for therapeutic fluid preparation, delivery, and volume management. More specifically, the invention relates to systems and methods utilizing non-invasive technologies and methodologies in the preparation of therapeutic fluid, the delivery of said fluid during treatment, as well as fluid volume or fluid weight management during dialysis and other similar blood treatment type therapies.

2. The Prior Art

Dialysis is a process for removing toxins and waste (through diffusion), and excess water (through ultrafiltration) from blood. It is primarily used to provide an artificial replacement for lost kidney function in people with renal failure. Dialysis may be used for those with an acute disturbance in kidney function (acute kidney injury or acute renal failure) or for those with progressive, but chronically worsening kidney function—a state known as chronic kidney disease stage 5 (chronic renal failure or end-stage kidney disease).

Dialysis works on the principles of the diffusion of solutes and ultrafiltration of fluid across a semi-permeable membrane that separates a blood compartment from a therapeutic fluid compartment within a dialyzer. Diffusion describes the movement of particles in fluids. Substances in water tend to move from an area of high concentration to an area of low concentration. The therapy of dialysis utilizes this phenomenon and moves specific particles from the blood (area of high concentration) into the therapeutic fluid compartment (area of low concentration).

A dialysis system generally includes two fluid paths, a blood tubing set (BTS) and a dialysate flow path that both communicate through a dialyzer. The BTS generally includes an arterial line for drawing blood from a patient and a venous line for returning blood to the patient. Similarly, the dialysate flow path has a line that takes fresh dialysate from a source, to the dialyzer and a line that removes “used” dialysate from the dialyzer and delivers it to drain or to a reservoir to be used again. Blood, in the blood extracorporeal circuit side of the system, flows through the BTS and the blood compartment of the dialyzer. The blood compartment of the dialyzer is comprised of small tubules known as hollow fibers that are made of a semipermeable membrane. Smaller solutes and fluid within the blood are able to pass through the semipermeable membrane and into the therapeutic fluid compartment of the dialyzer. The semipermeable membrane that makes up the hollow fibers within the dialyzer does not permit the passage of larger substances (for example, red blood cells, large proteins) into the therapeutic fluid compartment of the system.

There are many conventional systems and methods for dialysis, including hemodialysis (as described above), hemofiltration, hemodiafiltration, and similar blood treatment therapies.

In hemodialysis, a patient is connected to a blood extracorporeal circuit using needles, catheters, or fistulas that are inserted into the patient's veins and arteries. The patient's blood is pumped through the extracorporeal circuit on its way to the dialyzer. The hollow fibers made of a semipermeable membrane allow the blood filtering device to remove unwanted solutes and excess fluid from the blood into the therapeutic fluid. This is accomplished by flowing blood through the fibers in a counter-current direction to the dialysate flowing around the outside of the fibers. The cleansed blood is then returned via the extracorporeal blood circuit back to the body. Ultrafiltration (the process of fluid removal from the blood) is achieved by increasing the hydrostatic pressure across the dialyzer. This is usually accomplished by generating a negative pressure in the dialysate compartment of the dialyzer. This pressure gradient causes fluid and dissolved solutes to move from blood to dialysate. Prior to connecting a patient to a hemodialysis system, it is necessary to prime the sterile blood tubing set (BTS) and dialyzer. Priming of the BTS and dialyzer ensures that air is not returned to the patient during treatment.

Hemofiltration is a similar treatment to hemodialysis, but it makes use of a different principle. The blood is pumped through a dialyzer or hemofilter as in dialysis, but the therapeutic benefit is accomplished by continuous fluid removal and replacement, as opposed to through diffusion via concentration gradients. As in the process of ultrafiltration in hemodialysis, hemofiltration moves fluid from the blood into the therapeutic fluid side of the blood filter using a pressure gradient. As fluid moves out of the blood and into the therapeutic fluid it carries with it many dissolved substances, more importantly ones with large molecular weights, which are cleared less well by hemodialysis. Hemofiltration is more effective at large molecular weight solute removal because of the use of high flux dialyzers and the effect known as solute drag or convective removal. Due to the large amount of fluid processed a lot of ions and small-to-middle molecular weight molecules are lost from the blood and therefore must be returned to the body using a replacement fluid or substitution fluid during the treatment.

Hemodiafiltration is a term used to describe several methods of combining hemodialysis and hemofiltration in one process. The objective being combining the desirable attributes of both treatment modalities. That is, removing small molecules (as in hemodialysis), as well as larger molecules (as in hemofiltration).

Dialysis patients generally retain unwanted fluid. The process of dialysis not only reduces the blood concentration of certain undesirable molecules (e.g. urea, uric acid) it generally also removes excess blood volume to deal with the fluid retention. Traditional systems will capture a certain volume of fluid coming out of the therapeutic fluid circuit side of the system and weigh it. This fluid mass is above and beyond that which was delivered to the dialyzer, therefore, it is fluid that has made its way through the fibers from the blood extracorporeal circuit side of the system. This fluid removed from the patient is referred to as ultrafiltration fluid and it is typically programmed into the treatment. That is, there is a target volume to be removed at the start of almost every treatment.

Backflush is a procedure performed during a dialysis treatment to clean the inlet side of the dialyzer. That is, after a certain period of treatment time blood cells and platelets will begin to accumulate at the (blood) inlet to the dialyzer creating back-pressure against the blood pump and reducing blood flow through the dialyzer, if left unmanaged. Conventional dialysis systems will stop the forward rotation of the blood pump and reverse the flow, while simultaneously pushing dialysate through the dialyzer using an ultrafiltration pump. The ultrafiltration pump uses dialysate fluid collected in the ultrafiltration tank to perform the backflush. The amount of fluid used in the backflush is measured in the ultrafiltration tank.

In conventional systems and methods there is generally some dependency on relatively troublesome and costly, invasive contact with the therapeutic fluid during dialysis to confirm such things as conductivity, temperature, pressure and flow. For example, in conventional systems transmembrane pressure or TMP measurement is generally used to control therapeutic fluid movement through the semipermeable membrane of the dialyzer. The TMP is the difference between the average blood side pressures in the blood extracorporeal circuit and the average dialysate side pressures in the therapeutic fluid circuit. The TMP is conventionally measured by introducing invasive pressure measurement devices at the inlets and outlets of the dialyzer in both the blood extracorporeal circuit and the dialysate circuit.

The conventional invasive pressure measurement elements make it difficult to create a flow path and in general, a therapeutic solution that is relatively cost-effective and trouble-free. For example, the conventional invasive pressure measurement elements increase cost, introduce complexity, sterility and maintenance problems. Accordingly, there is an unmet need for therapeutic systems and methods to minimize the dependency on invasive contact with therapeutic fluid during dialysis.

In addition, the raw components of therapeutic fluid, as well as the final mixed therapeutic fluid, are generally confirmed by either conductivity, pH measurements, or both, which are also measured by introducing invasive sensors into the therapeutic fluid preparation systems. These invasive elements also make it difficult to create a cost-effective therapeutic fluid circuit. Accordingly, there is an unmet need of delivering a cost-effective therapy using minimally invasive technologies and methodologies.

### SUMMARY OF THE INVENTION

The present invention provides therapeutic systems and methods for dialysis and similar fluid treatment therapies, as well as for the preparation of the necessary therapeutic fluid.

One aspect of the present invention systems and methods minimizes the dependency on invasive contact with therapeutic fluid during the preparation of therapeutic fluid by utilizing non-invasive fluid volume or fluid weight monitoring during the preparation of therapeutic fluid.

In accordance with one aspect of the invention, a system and method is provided for fluid volume or fluid weight measurement of therapeutic fluid during the preparation of therapeutic fluid. A therapeutic fluid source includes a first therapeutic fluid component having a first known mass provided in a first fluid container and a second therapeutic fluid component having a second known mass provided in a second fluid container. The first therapeutic fluid component and the second therapeutic fluid component are in fluid communication with each other. The first therapeutic fluid container and the second therapeutic fluid container are in cooperative engagement with a weight measuring system. The weight measuring system includes a first load cell, a second load cell, and a connected strain gauge which monitors the fluid volume or fluid weight in the first fluid container and the second fluid container. A first peristaltic pump cooperatively engages a first tube in fluid communication between the first fluid container and a first end of a filter bypass. A second peristaltic pump cooperatively engages a second tube in fluid communication between the second fluid container and a second end of the filter bypass. The first peristaltic pump and the second peristaltic pump are configured to pump the first therapeutic fluid component and the second therapeutic fluid component between the first fluid container and the second fluid container. The first load cell, the second load cell, and the connected strain gauge are engaged by a weight control scheme programmed to operate the first peristaltic pump and the second peristaltic pump. The weight control scheme includes a feedback loop from the first load cell, the second load cell, and the connected strain gauge to the first peristaltic pump and the second peristaltic pump to facilitate mixing of the first therapeutic fluid component and the second therapeutic fluid component to form a treatment ready therapeutic fluid.

In operation, the system and method confirms adequate mixing of the first therapeutic fluid component and the second therapeutic fluid component by measuring the distribution of fluid weight or fluid volume. That is, it measures the fluid weight or fluid volume of the first fluid container, the second fluid container, and both collectively.

In accordance with one aspect of the invention, the first load cell and the second load cell are each provided with a keyed mechanism which only allows connection to the corresponding first fluid container or second fluid container.

To facilitate blood filtration, once the therapeutic fluid has been adequately mixed, the first tube to the filter bypass and the second tube to the filter bypass can be clamped and the filter bypass can be removed. In its place is installed the appropriate blood filtering device (e.g. a conventional dialyzer) with a connected extracorporeal circuit. The first tube and second tube are then unclamped and the therapeutic fluid is circulated from the therapeutic fluid circuit side of the system through the dialyzer in order to prime the dialyzer and a blood extracorporeal circuit side of the blood therapy system.

Another aspect of the invention provides a blood therapy system and method for continuous weight or volumetric measurement of therapeutic fluid from a therapeutic fluid source during dialysis, which minimizes the need for introducing invasive monitoring devices in the flow path. A therapeutic fluid source is in cooperative engagement with a weight measuring system which monitors the fluid volume or fluid weight of the therapeutic fluid in the therapeutic fluid source. The first peristaltic pump cooperatively engages a first tube in fluid communication between a conventional dialyzer (or blood filtering device) and the therapeutic fluid source. The second peristaltic pump cooperatively engages a second tube in fluid engagement between the dialyzer (or blood filtering device) and the therapeutic fluid source. The first peristaltic pump and the second peristaltic pump are configured to pump therapeutic fluid from the therapeutic fluid source. The first peristaltic pump and the second peristaltic pump are also configured to pump therapeutic fluid from the therapeutic fluid circuit side of the blood therapy system to or from the dialyzer. The weight measuring system is engaged by a weight control scheme programmed to operate the first peristaltic pump and the second peristaltic pump. The weight control scheme includes a feedback loop from the weight measuring system to the first peristaltic pump and the second peristaltic pump to indirectly monitor and control of the TMP of the dialyzer.

In operation, a pressure imbalance at the dialyzer causes a change in the volume or weight of the therapeutic fluid in the therapeutic fluid source. More specifically, a pressure imbalance at the dialyzer causes a change in the rate of volume or weight gain (or loss) in the therapeutic fluid source, which when detected can be adjusted by the combined flow rates through the first peristaltic pump and the second peristaltic pump. If the first peristaltic pump is delivering therapeutic fluid to the dialyzer faster than the second peristaltic pump is removing therapeutic fluid from the dialyzer, the fluid pressure caused by the excess therapeutic fluid supplied by the first peristaltic pump will increase and therapeutic fluid will move through the semipermeable membrane fibers of the dialyzer and into the blood. If the second peristaltic pump is removing therapeutic fluid from the dialyzer faster than the first peristaltic pump is delivering therapeutic fluid to the dialyzer, the drop in therapeutic fluid pressure across the dialyzer caused by the excess demand of the second peristaltic pump will result in blood volume moving through the semipermeable membrane fibers of the dialyzer and into the therapeutic fluid. In either case, the pressure imbalance will be detectable by the changing volume or weight in the therapeutic fluid source. As should readily be understood, the first peristaltic pump and the second peristaltic pump can maintain the pressure imbalance virtually at zero (if that is desired) by ensuring that the rate of volume or weight change is virtually zero throughout the therapy.

Another aspect of the present invention systems and methods provides an extracorporeal blood circuit with the venous line and the arterial line removably connected to each other in fluid communication by a recirculation connector line for the purposes of priming the BTS (inclusive of the dialyzer) through the dialyzer. After priming, removal of a recirculation connector line allows for connection of the venous line and the arterial to needles, catheters, or fistulas, which ultimately enable connection to the patient.

In operation, priming of the blood extracorporeal circuit side of the system is accomplished by utilizing the first peristaltic pump and the second peristaltic pump to elevate pressure in the therapeutic fluid flow path between the first peristaltic pump and the second peristaltic pump, thereby, increasing the therapeutic fluid pressure across the dialyzer and resulting in fluid flow from the therapeutic fluid circuit side through the semipermeable membrane fibers of the dialyzer and into the blood extracorporeal circuit side of the blood therapy system. Differential flows between first peristaltic pump and the second peristaltic pump will force therapeutic fluid from the therapeutic fluid circuit side of the blood therapy system, through the semipermeable membrane fibers of the dialyzer, and into the blood extracorporeal circuit side of the blood therapy system. The first peristaltic pump and the second peristaltic pump may also increase pressure in the flow path between the first peristaltic pump and the second peristaltic pump by pumping directly toward the dialyzer simultaneously, resulting in therapeutic fluid moving from the therapeutic fluid circuit side of the blood therapy system, through the dialyzer fibers, and into the blood extracorporeal circuit side of the blood therapy system. With a peristaltic blood pump (in the blood extracorporeal circuit side of the blood therapy system) moving in alternating forward and backward directions, therapeutic fluid can be directed throughout the blood extracorporeal circuit side of the blood therapy system to complete the priming. The completion of priming is confirmed when the weight or volume of the therapeutic fluid in the therapeutic fluid source substantially reaches equilibrium, air in-line sensors both detect fluid, and the lower level sensor of an air trap senses liquid. With the entire blood therapy system primed, therapy can be initiated by clamping the arterial and venous lines, removing the recirculation connector in the blood extracorporeal circuit side of the blood therapy system, connecting the venous line and the arterial line to needles, catheters, or fistulas which are connected to the patient. Finally, prior to commencing the therapy the arterial and venous lines are again unclamped.

In another aspect of the present invention, the first therapeutic fluid component and the second therapeutic fluid component are substituted for pre-mixed treatment-ready therapeutic fluid.

Another aspect of the present invention systems and methods provides an extracorporeal blood circuit with the venous line and the arterial line removably connected via removable tubes to the therapeutic fluid source for the purposes of priming the BTS. After priming, disconnection of the venous line and the arterial line from the therapeutic fluid source allows for connection of the venous line and the arterial line to needles, catheters, or fistulas, which ultimately enable connection to the patient.

In operation, priming of the blood extracorporeal circuit side of the blood therapy system from the therapeutic fluid source is accomplished by utilizing the first peristaltic pump and the second peristaltic pump to elevate pressure in the flow path between the first peristaltic pump and the second peristaltic pump, resulting in fluid flow from the therapeutic fluid circuit side of the blood therapy system, through the semipermeable membrane of the dialyzer fibers, and into the blood extracorporeal circuit side of the blood therapy system. Differential flows between the first peristaltic pump and the second peristaltic pump resulting in increased therapeutic fluid pressure across the dialyzer will force therapeutic fluid from the therapeutic fluid circuit side of the blood therapy system, through the semipermeable membrane of the dialyzer fibers, and into the blood extracorporeal circuit side of the blood therapy system. The first peristaltic pump and the second peristaltic pump may also increase pressure in the flow path between the first peristaltic pump and the second peristaltic pump by pumping directly toward the dialyzer simultaneously, resulting in therapeutic fluid moving from the therapeutic fluid circuit side of the blood therapy system, through the semipermeable membrane of the dialyzer fibers, and into the blood extracorporeal circuit side of the blood therapy system. With a third peristaltic pump in the blood extracorporeal circuit side of the blood therapy system rolling in the backward direction (i.e. toward the arterial line), therapeutic fluid can be directed through the arterial line and back toward the therapeutic fluid source. With a third peristaltic pump in the blood extracorporeal circuit side of the blood therapy system in the forward direction, therapeutic fluid can be directed toward the air trap and down the venous line on its way back to the therapeutic fluid source. The venous line may also be primed by stopping the third peristaltic pump and opening the venous line clamp. The therapeutic fluid being forced through the semipermeable membrane fibers of the dialyzer is moved into the air trap, through the venous line, and into the therapeutic fluid source. The completion of priming is confirmed when the weight or volume of the therapeutic fluid in the therapeutic fluid source substantially reaches equilibrium, the air in-line sensors both detect fluid, and the lower level sensor and upper level sensor of the air trap indicates a liquid level there between. With the entire blood therapy system primed, therapy can be initiated by removing the removable tubes to allow for connection of the venous line and the arterial line to needles, catheters, or fistulas, which ultimately enable connection to the patient.

Another aspect of the present invention systems and methods provides a method of providing blood therapy including the steps of providing a blood tubing set and a therapeutic fluid source supplying a therapeutic fluid, the blood tubing set and the therapeutic fluid source separated by a blood filtering device having a transmembrane pressure, providing a first tube in fluid communication between the therapeutic fluid source and the blood filtering device, providing a second tube in fluid communication between the therapeutic fluid source and the blood filtering device, providing a first peristaltic pump in cooperative engagement with the first tube, providing a second peristaltic pump in cooperative engagement with the second tube, providing a third peristaltic pump in cooperative engagement with the blood tubing set, the third peristaltic pump having a first pressure sensor in cooperative engagement with the inlet of the third peristaltic pump and a second pressure sensor in cooperative engagement with the outlet of the third peristaltic pump, programming a pressure control scheme having a feedback loop cooperatively engaged with the first peristaltic pump, the second peristaltic pump, and the third peristaltic pump to monitor and control the transmembrane pressure without invasively contacting the therapeutic fluid.

Another aspect of the present invention systems and methods provides ultrafiltration using the systems and devices described above.

In operation, the combined flow rates through the first peristaltic pump and the second peristaltic pump are controlled to produce a net fluid removal from the blood extracorporeal circuit side of the blood therapy system (ultimately coming from the patient) during the blood treatment. Net fluid removal from the patient is monitored by the weight or volume of the therapeutic fluid in the therapeutic fluid source, thereby eliminating the need for dedicated pumps or hardware to perform ultrafiltration.

Another aspect of the present invention systems and methods provides backflush of the blood therapy device during treatment using the systems and devices described above.

In one embodiment, the combined flow rates through the first peristaltic pump and the second peristaltic pump produces a net fluid addition to the blood extracorporeal circuit side of the blood therapy system ultimately transferred to the patient. The net fluid transfer to the patient is monitored by the change in weight or volume of the therapeutic fluid in the therapeutic fluid source, thereby eliminating the need for dedicated pumps or hardware to separately perform backflush and allowing backflush during dialysis.

The above aspects may be performed alone or in combination with the others.

It is therefore an object of the invention to provide systems and methods which minimize the dependency on invasive contact with therapeutic fluid during the preparation of therapeutic fluid by utilizing non-invasive fluid volume or fluid weight monitoring during the preparation of therapeutic fluid.

It is another object of the present invention to provide systems and methods which minimize the dependency on invasive contact with therapeutic fluid during dialysis and similar fluid treatment therapies by utilizing non-invasive fluid volume or fluid weight monitoring during dialysis.

It is yet another object of the present invention to provide improved therapeutic fluid systems and methods that perform backflush during dialysis.

It is yet another object of the present invention to provide improved therapeutic fluid systems and methods that perform priming before dialysis.

It is yet another object of the present invention to provide improved therapeutic fluid systems and methods that perform ultrafiltration in connection with dialysis.

It is a further object of the present invention to provide improved therapeutic fluid systems and methods that provide simplified flow regimes in connection with dialysis.

It is still a further object of the present invention to provide improved therapeutic fluid systems and methods that reduce complexity, sterility and maintenance problems by minimizing the dependency on invasive contact with therapeutic fluid during dialysis and similar fluid treatment therapies by utilizing non-invasive fluid volume or fluid weight monitoring during dialysis.

These together with other objects of the present invention, along with the various features of novelty which characterize the present invention, are pointed out with particularity in the claims annexed to and forming a part of this disclosure. For a better understanding of the present invention, its operating advantages and the specific objects attained by its uses, reference should be had to the accompanying drawings and descriptive matter in which there is illustrated a preferred embodiment of the present invention and alternative embodiments.

Before explaining the preferred embodiment and alternative embodiments of the present invention in detail, it is to be understood that the present invention is not limited in its application to the details of construction, to the arrangements of the components set forth in the following description or illustrated in the drawings, or to the methods described therein. The present invention is capable of other embodiments and of being practiced and carried out in various ways. Also, it is to be understood that the phraseology and terminology employed herein are for the purpose of description and should not be regarded as limiting.

There has thus been outlined, rather broadly, the more important features of the invention in order that the detailed description thereof that follows may be better understood, and in order that the present contribution to the art may be better appreciated. There are, of course, additional features of the invention that will be described hereinafter and that will form the subject matter of the invention.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the present invention in any way.

### BRIEF DESCRIPTION OF THE DRAWINGS

FIG. 1 is a schematic of a conventional blood extracorporeal circuit with dialyzer.

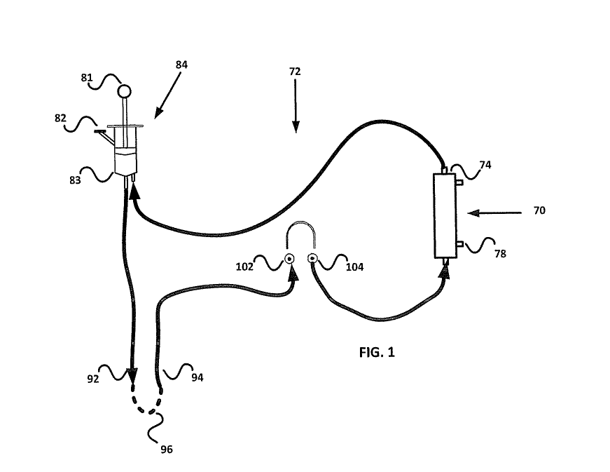

FIG. 2 is an illustration of a system for the preparation of therapeutic fluid in accordance with the invention.

FIG. 3 is an illustration of the system in FIG. 2 with front doors removed in order to demonstrate a first therapeutic fluid component and a second therapeutic fluid component in accordance with the invention.

FIG. 4 is an illustration of a blood therapy system for dialysis in a configuration arrangement intended for the priming of the blood extracorporeal circuit with previously mixed and heated therapeutic fluid in accordance with the invention.

FIG. 5 is an illustration of a completely primed blood therapy system for dialysis which is ready for connection of both venous and arterial patient fistulas to the patient in accordance with the invention.

FIG. 6 is an illustration of a blood therapy system in treatment mode (i.e. patient connected) with virtually zero transmembrane pressure in accordance with the invention.

FIG. 7 is an illustration of a blood therapy system in treatment mode with a greater average blood side pressure than an average therapeutic fluid side pressure resulting in ultrafiltration in accordance with the invention.

FIG. 8 is an illustration of a blood therapy system in treatment mode with a greater average therapeutic fluid side pressure than an average blood side pressure and a reversed flow third peristaltic pump resulting in backflush or back-filtration in accordance with the invention.

FIG. 9 is an illustration of a blood therapy system in treatment mode with a greater average therapeutic fluid side pressure than an average blood side pressure resulting in patient solution infusion in accordance with the invention.

FIG. 10 is an illustration of a blood therapy system in a treatment mode referred to as single needle dialysis in accordance with the invention.

FIG. 11 is an illustration of the dialyzer bypass containing a first metal probe and a second metal probe for measuring fluid conductivity during fluid preparation in accordance with the invention.

FIG. 12 is an illustration of the preferred recirculation connector for priming of a conventional blood extracorporeal circuit in accordance with the invention.

FIG. 13 is an illustration of the alternate recirculation connector for priming of a blood extracorporeal circuit designed for the purposes of single needle dialysis

FIG. 14 is exploded view of a dialyzer (or blood filtering device) taken along line 14-14 of FIG. 4.

FIG. 15 is a chart demonstrating the target fluid removal profile line (ultrafiltration profile) for a prescribed therapy and the consequences of blood extracorporeal circuit occlusions in accordance with the invention.

FIG. 16 depicts a syringe style blood air trap.

FIG. 17 displays the blood tubing set prior to being integrated with the rest of the blood extracorporeal circuit.

### DETAILED DESCRIPTION OF THE PREFERRED EMBODIMENT

While this present invention is susceptible of embodiments in many different forms, there are shown in the drawings and will be described in detail herein, a preferred embodiment, with like parts designated by like reference numerals and with the understanding that the present disclosure is to be considered as an exemplification of the principles of the present invention, and is not intended to limit the claims to the illustrated preferred embodiment.

### Preparation of Therapeutic Fluid & Priming of Therapeutic Fluid Circuit

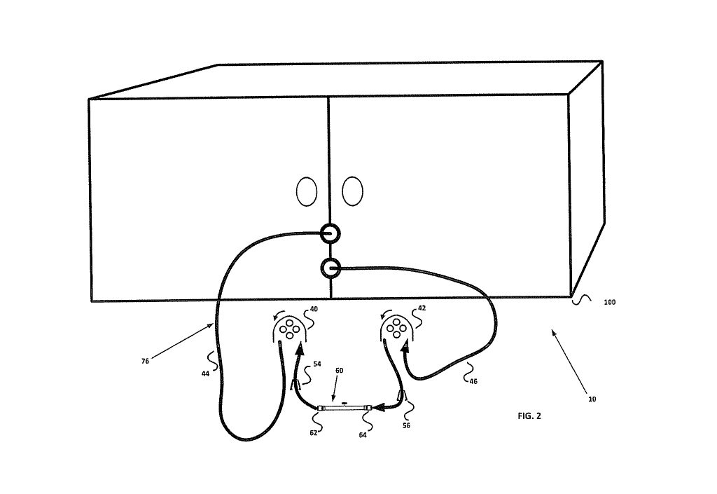

Referring now to FIG. 2, a system 10 for the preparation of therapeutic fluid is illustrated. System 10 includes a therapeutic fluid warming and fluid volume measuring chamber 100. In the preferred embodiment, the thermally insulated fluid warming chamber 100 contains a low power heater.

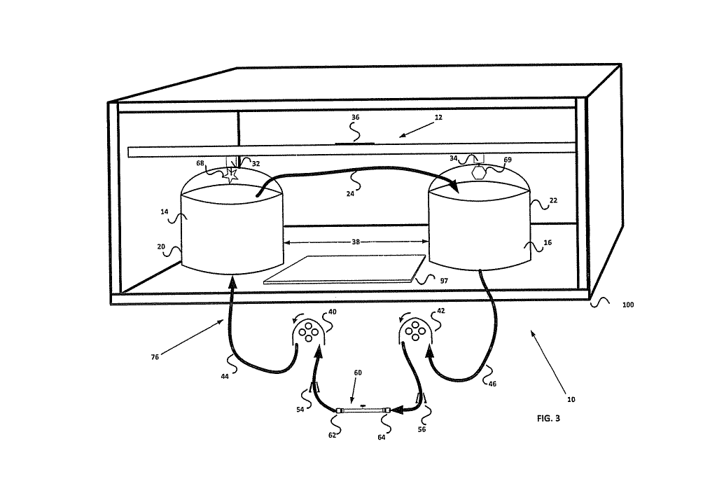

As illustrated in FIG. 3, showing the therapeutic fluid warming and fluid volume measuring chamber 100 with doors removed. System 10 utilizes a non-invasive fluid volume or fluid weight monitoring system 12 during the preparation of therapeutic fluid 39 (not shown). A therapeutic fluid source 38 includes a first therapeutic fluid component 14 (not shown) having a first known mass provided in a first fluid container 20 and a second therapeutic fluid component 16 (not shown) having a second known mass provided in a second fluid container 22.

In the preferred embodiment, the first therapeutic fluid component 14 and the second therapeutic fluid component 16 are in fluid communication with each other via a fluid communication device 24 to facilitate mixing thereof. The fluid communication device 24 is a sterile double-ended spike, although it should be readily understood by those skilled in the art that other suitable fluid communication devices may be employed.

The first therapeutic fluid container 20 and the second therapeutic fluid container 22 are in cooperative engagement with a weight monitoring system 12. In the preferred embodiment, the weight monitoring system 12 includes a first load cell 32 which monitors the fluid volume or fluid weight in the first fluid container 20, a second load cell 34 which monitors the fluid volume or fluid weight in the second fluid container 22, and a connected strain gauge 36 which monitors the fluid volume or fluid weight in the first fluid container 20 and the second fluid container 22 collectively, although it should be readily understood by those skilled in the art that other suitable non-intrusive weight monitoring systems and devices may be employed.

In the preferred embodiment, a first peristaltic pump 40 cooperatively engages a first tube 44 in fluid communication between the first fluid container 20 and a first end 62 of a filter bypass 60. A second peristaltic pump 42 cooperatively engages a second tube 46 in fluid communication between the second fluid container 22 and a second end 64 of the filter bypass 60. The first peristaltic pump 40 and the second peristaltic pump 42 are configured to pump the first therapeutic fluid component 14 and the second therapeutic fluid component 16 between the first fluid container 20 and the second fluid container 22 for the purposes of priming, mixing and heating. The first load cell 32, the second load cell 34, and the connected strain gauge 36 are engaged by a weight control scheme (not shown) programmed to operate the first peristaltic pump 40 and the second peristaltic pump 42.

The weight control scheme (not shown) includes a feedback loop from the first load cell 32, the second load cell 34, and the connected strain gauge 36 to the first peristaltic pump 40 and the second peristaltic pump 42 to facilitate mixing of the first therapeutic fluid component 14 and the second therapeutic fluid component 16 to form a therapeutic fluid 39 and to prime the system 10 with a heated (provided by heating element 97) therapeutic fluid 39.

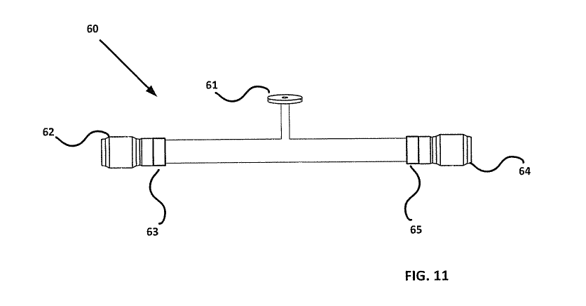

In operation, the system 10 and method confirms adequate mixing of the first therapeutic fluid component 14 and the second therapeutic fluid component 16 by measuring the distribution of fluid weight or fluid volume in the first fluid container 20, the second fluid container 22, and both. As an example, if the second fluid container 22 is a 5 (five) liter container and contains 5 (five) liters of bicarbonate solution, the contents of which are controlled in manufacturing, and the first fluid container 20 is a 5 (five) liter container, but only contains 0.5 (one half) liter of acid, the contents of which are also controlled in manufacturing, then by simply monitoring the weight distribution in the first fluid container 20, the second fluid container 22, and both, the system 10 can confirm adequate mixing to form a therapeutic fluid 39 (not shown). In greater detail, if the 5 (five) liters of fluid in the second fluid container 22 are moved toward the 5 (five) liter first fluid container 20 which only contains 0.5 (half) of a liter of fluid, then the weight of the second fluid container 22 will continue to decline at the equal rate of increase of the first fluid container 20. Furthermore, when the second fluid container 22 has moved 4.5 liters of fluid into the first fluid container 20, the first fluid container 20 will begin to spill over into the second fluid container 22 via the fluid communication device 24. Once this state is achieved, recirculating flow can be continued to adequately heat (using heating element 97) and mix the first therapeutic fluid 14 and the second therapeutic fluid component 16 resulting in a final batch of 5.5-L of therapeutic fluid 39 (not shown) that is warmed to body temperature. Adequate heating and mixing can also be achieved by reversing flow and taking fluid from the first fluid container 20 to the second fluid container 22 and the same sequence can be observed, as described above. How many iterations would be needed to accomplish adequate heating and mixing will depend on the empirical data and could be confirmed by a safety monitoring conductivity measurement via a first metal probe 63 and a second metal probe 65 in the dialyzer bypass 60 (FIG. 11). In the preferred embodiment, first end 62 of the dialyzer bypass 60 and the second end 64 of the dialyzer bypass 60 comprise Hansen ports for removable connection to therapeutic fluid circuit 76 for the purposes of therapeutic fluid 39 preparation prior to priming of the blood extracorporeal circuit 72. In operation, first metal probes 63 and second metal probe 65 in the dialyzer bypass 60 permit therapeutic fluid 39 contact within the dialyzer bypass 60 and electrical engagement to the weight monitoring system 12 allowing the passage of a small current through the first metal probe 63, then through therapeutic fluid 39 and finally returning to the weight monitoring system 12 for resistivity measurement via metal second probe 65, thereby confirming completion of mixing. It should be readily understood by those skilled in the art that three or more fluid containers and corresponding fluid components may be employed.

In the preferred embodiment, the first load cell 32 and the second load cell 34 are each provided with keyed mechanisms 68 and 69, respectively, such as a hook, hanger, boss, or the like, which only allow connection to the corresponding first fluid container 14 or second fluid container 16.

### Priming of the Blood Extracorporeal Circuit with a Recirculation Connector

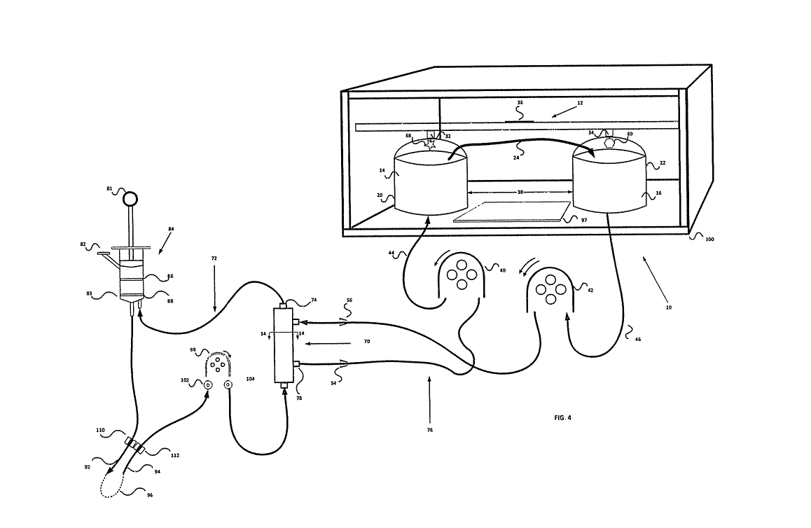

As shown in FIG. 4, to facilitate dialysis, once the therapeutic fluid 39 (not shown) has been adequately heated and mixed, the first tube 44 to the filter bypass 60 and the second tube 46 to the filter bypass 60 can be clamped via clamps 54 and 56, respectively, in order to reduce fluid loss during the manual substitution of the filter bypass 60 with a dialyzer 70 of a blood therapy system 15 having a therapeutic fluid circuit 76 and a blood extracorporeal circuit 72. The preferred embodiment illustrates the use of a dialyzer 70, although it should be readily understood by those skilled in the art that any suitable blood filtering device may be substituted for the dialyzer.

In the preferred embodiment, the dialyzer 70 enables the primed therapeutic fluid circuit 76 to communicate with the unprimed extracorporeal blood circuit 72. More specifically, the blood compartment 74 of a dialyzer 70 is in fluid communication with the therapeutic fluid compartment 78 of a dialyzer 70 via the semipermeable membrane hollow fibers 77.

In the preferred embodiment, a blood compartment 74 of a dialyzer 70 is in fluid communication with a blood extracorporeal circuit 72 having a blood tubing set 73. The therapeutic fluid compartment 78 of the dialyzer 70 is in fluid communication with the therapeutic fluid circuit 76. The blood compartment 74 of the dialyzer 70 and the therapeutic fluid compartment 78 of the dialyzer 70 are separated by the walls of semipermeable membrane hollow fibers 77. A third peristaltic pump 98 cooperatively engages the blood tubing set 73 of the blood extracorporeal circuit 72. A first pressure sensor 102 is in cooperative engagement with the inlet of third peristaltic pump 98. A second pressure sensor 104 is in cooperative engagement with the outlet of third peristaltic pump 98.

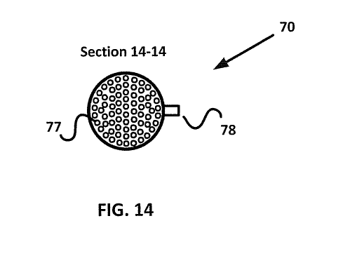

As illustrated in FIG. 14, a dialyzer 70 contains semipermeable membrane hollow fibers 77, the interior of which is the blood compartment 74 of the dialyzer 70 which makes up part of the blood extracorporeal circuit 72 and the surrounding space within the dialyzer 70 which is the therapeutic fluid compartment 78 of the dialyzer 70 which makes up part of the therapeutic fluid circuit 76.

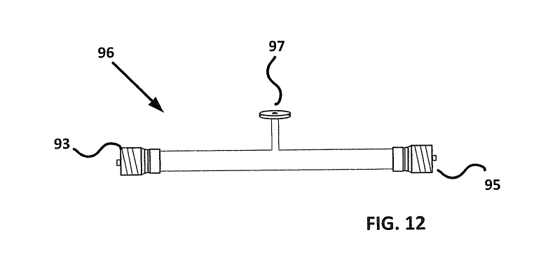

In operation, the replacement of the filter bypass 60 with the dialyzer 70 connected to the blood extracorporeal circuit 72 containing a recirculation connector 96 (FIG. 12), allows for the primed therapeutic fluid circuit 76 to be unclamped via clamps 54 and 56 and used to prime the blood extracorporeal circuit 72. The recirculation connector 96 is removably connected via luer thread connectors 93 and 95 to the arterial line 94 and venous line 92 for the purposes of priming the blood extracorporeal circuit 72. The recirculation connector 96 is equipped with a microbial membrane filtered vent line 97 for permitting the escape of air, but not therapeutic fluid 39 during the priming process. During fluid priming, air is purged from the blood extracorporeal circuit 72 through the removably connected recirculation connector 96 containing hydrophobic filter 97 which permits the escape of air, but not fluid. Similarly, air trap 84 can be manipulated to assist in the removal of air as the blood extracorporeal circuit 72 is primed with therapeutic fluid 39. Piston 81 of the syringe style blood air trap 84 can be raised beyond the microbial membrane protected vent line port 82 in order for the therapeutic fluid level to rise. Once the level of the therapeutic fluid 39 in the air trap 84 reaches sensor 86, the piston 81 can be lowered below the membrane protected vent line port 82.

In operation, a transmembrane pressure or pressure imbalance at the blood dialyzer 70 (between the blood side pressure and the therapeutic fluid side pressure) causes a change in the volume or weight of the therapeutic fluid 39 in the therapeutic fluid containers 20 and 22 collectively, which is detected by the weight monitoring system 12.

If the first peristaltic pump 40 is delivering therapeutic fluid 39 to the dialyzer 70 faster than the second peristaltic pump 42 is removing therapeutic fluid 39 from the dialyzer 70, the therapeutic fluid side pressure caused by the therapeutic fluid 39 supplied by the first peristaltic pump 40 will increase and therapeutic fluid 39 will move across the semi-permeable membranes of the hollow fibers in the dialyzer 70 and into the blood extracorporeal circuit 72.

Completion of priming the extracorporeal blood circuit 72 with therapeutic fluid 39 is verified by the weight monitoring system 12 and the detection of fluid at all of the air sensors 110, 112, 84 and 86 on the blood tubing set 73. The weight monitoring system 12 will no longer demonstrate a decline in volume of therapeutic fluid 39 in containers 20 and 22 as pumps 40, 42, and 98 continue to circulate therapeutic fluid volume 39 throughout the therapeutic fluid circuit 76 and the blood extracorporeal circuit 72.

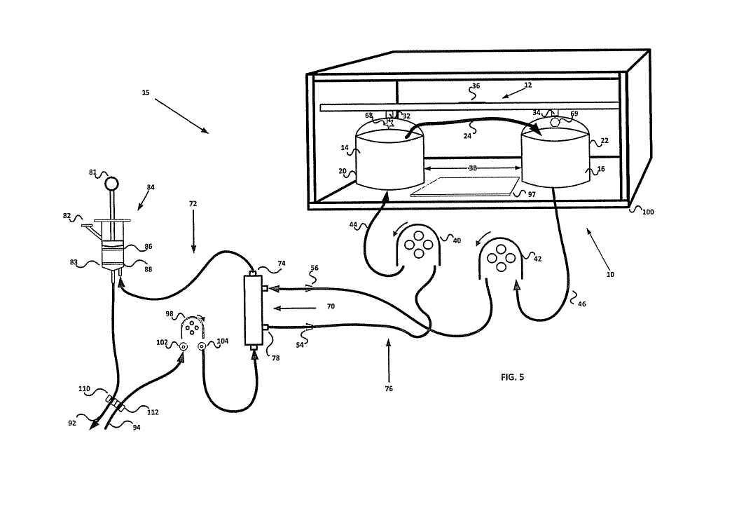

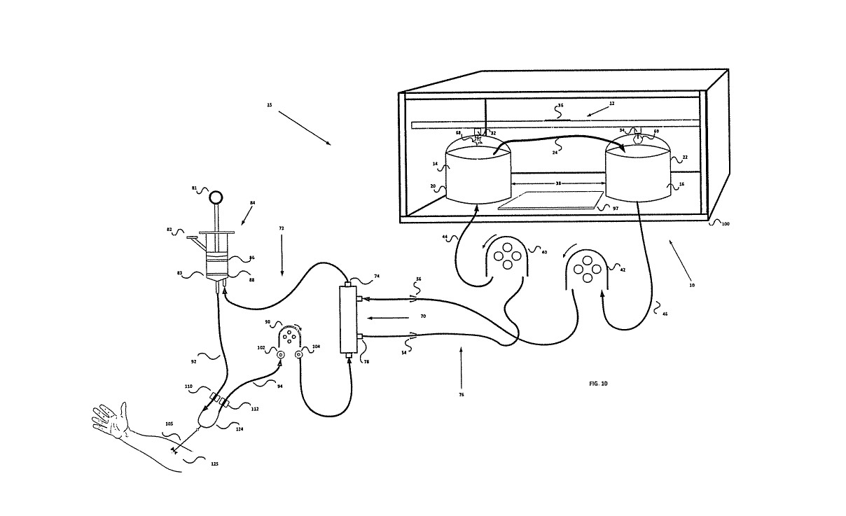

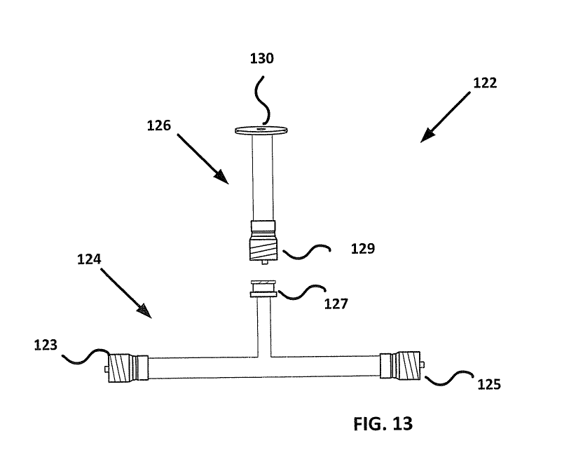

Referring now to FIG. 5, with both the therapeutic fluid circuit 76 and the blood extracorporeal circuit 72 primed, the patient can then be connected by first clamping the arterial line 94 and venous line 92 (to minimize fluid loss during removal of the recirculation connector 96), then removing the recirculation connector 96 (FIG. 12) in the blood extracorporeal circuit 72 to allow fluid communication between the venous line 92 and the arterial line 94 and needles, catheters, or fistulas which are ultimately connected to the patient. In the single needle configuration (FIG. 10) item 126 is removed from alternate recirculation system 122 (FIG. 13), leaving behind segment 124 for using during the treatment. Alternate recirculation system 122 (FIG. 13) is removably connected via luer thread (connectors 123 and 125) to the arterial line 94 and the venous line 92. Alternate recirculation connector 122 permits the fluid communication of the arterial line 94 to venous line 92 for the purposes of priming. During fluid priming, air is purged from the blood extracorporeal circuit 72 through the removably connected segment 126 containing hydrophobic filter 130 which permits the escape of air, but not fluid. Once the blood extracorporeal circuit 72 is completely primed, segment 126 can be removed and replaced by a single fistula needle 105 for the purposes of conducting a single needle therapy. Portion 124 remains connected for the duration of the treatment. The presence of portion 124 retains fluid communication between the arterial line 94 and the venous venous line 92 throughout the treatment. It also enables the connection of a single patient fistula to the blood extracorporeal circuit 72, thereby enabling fluid communication between said blood extracorporeal circuit 72 and the patient in a single needle format. Blood treatment can then begin (as set forth above) once the arterial line 94 and venous line 92 are unclamped.

### Blood Therapy State

### Fluid Equilibrium (0 TMP)

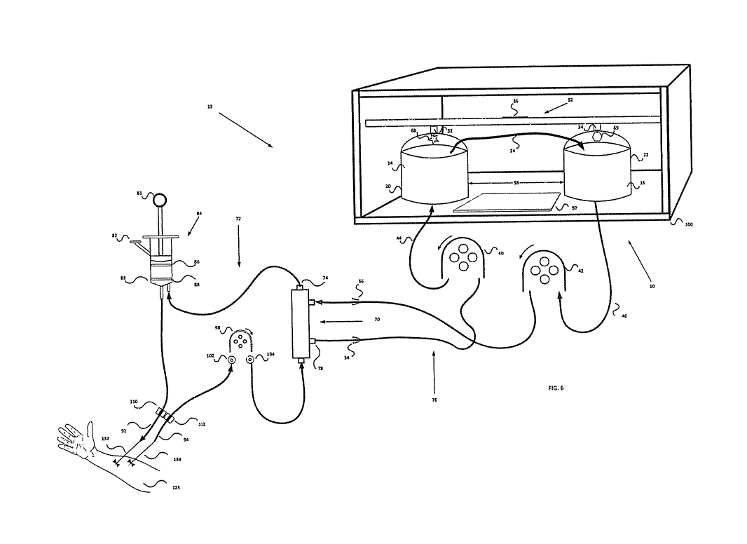

FIG. 6 demonstrates the therapy state of fluid equilibrium. That is, first peristaltic pump 40 removes virtually the same amount of the therapeutic fluid 39 delivered by second peristaltic pump 42 to the dialyzer 70. This state of fluid equilibrium results in virtually no net therapeutic fluid 39 volume gain by the patient 125, nor a patient blood volume reduction and is monitored by therapeutic volume measurement system 12. The comparison of load cells 32 and 34 confirms the therapy state of fluid equilibrium by verifying that the fluid volume decline in container 22 is virtually the same as the fluid volume increase in container 20. This state of fluid equilibrium allows unwanted waste solute in the blood to be diffuse into the therapeutic fluid 39 through the semipermeable membrane hollow fibers 77 of the dialyzer 70 without the usual accompaniment of fluid removal (ultrafiltration).

### Blood Therapy State

### Ultrafiltration

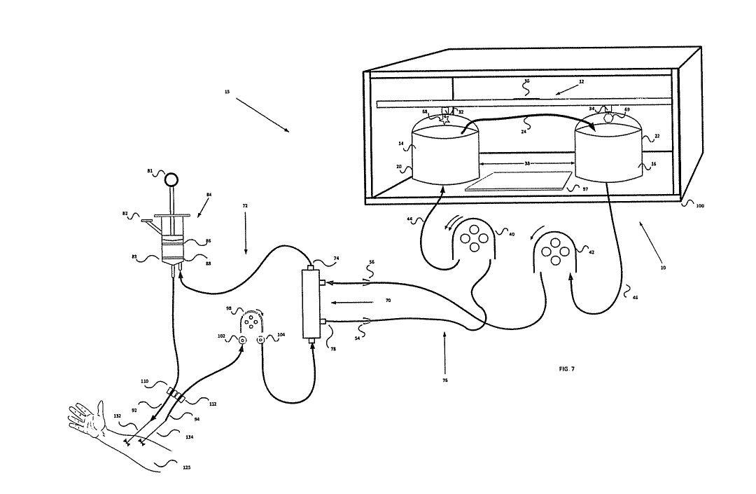

As shown in FIG. 7, the combined flow rates through the first peristaltic pump 40 and the second peristaltic pump 42 may be controlled to produce a net fluid removal from the blood extracorporeal circuit 72 which is monitored by the weight or volume of the therapeutic fluid 39 in the therapeutic fluid source containers 20 and 22, thereby eliminating the need for dedicated pumps or hardware to perform ultrafiltration. The rate of fluid volume increase in container 20 is virtually the same as the rate of fluid volume decrease in container 22, plus the desired fluid removal rate from the patient 125.

### Blood Therapy State

### Backflush

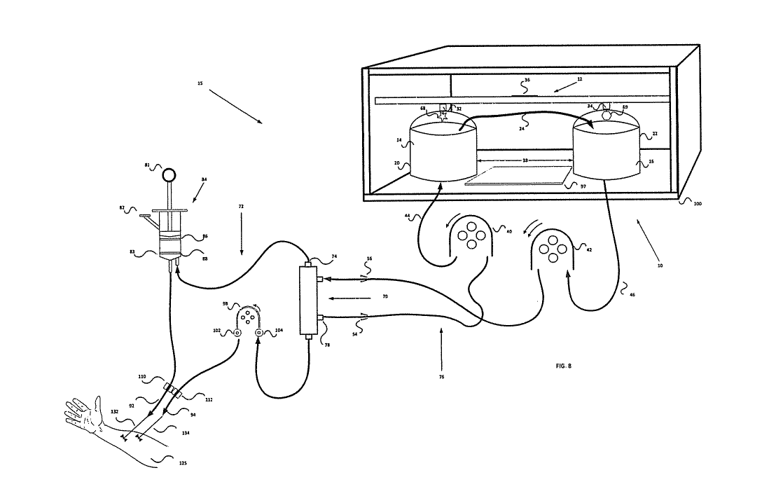

As shown in FIG. 8, the combined flow rates through the first peristaltic pump 40 and the second peristaltic pump 42 produces a net therapeutic fluid loss from container 22 and a corresponding fluid gain by the patient 125. This effect working in conjunction with a reversed flow of third peristaltic pump 98 will aid in the reduction of coagulated or congealed blood at the blood inlet side of the dialyzer 70. The rate of fluid volume increase in container 20 is at the rate of fluid volume decrease in container 22, less the desired backflush fluid rate which is virtually the same as the rate of fluid volume gain for the patient. The desired backflush fluid rate and the frequency of backflushes throughout the duration of the blood treatment is highly patient dependent and would be determined through empirical data. This fluid is usually accounted for by the therapy system and is removed gradually throughout the treatment in addition to the desired ultrafiltration.

### Blood Therapy State

### Solution Infusion

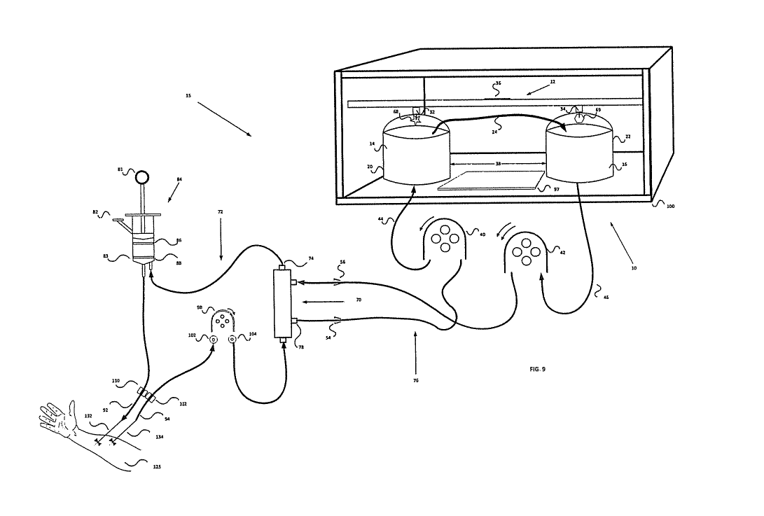

FIG. 9 demonstrates the combined flow rates through the first peristaltic pump 40 and the second peristaltic pump 42 producing a net therapeutic fluid loss from container 22 and a corresponding fluid gain by patient 125 This concert of events during treatment is intended to address instantaneous patient requests for therapeutic fluid 39 boluses to address potential hypotensive moments. It should be readily understood by those skilled in the art that Solution Infusion can be achieved with the third peristaltic pump 98 moving in the forward (clockwise) or reverse (counterclockwise) directions or some combination thereof.

### Blood Therapy State

### Hemodiafiltration

It should be readily understood by those skilled in the art that Blood Therapy States mentioned above (Ultrafiltration, Backflush and/or Solution Infusion) can be utilized in shorter but more aggressive rates in order to achieve the desired effect of Hemodiafiltration.

### Blood Therapy State

### Fluid Line Occlusion Detection

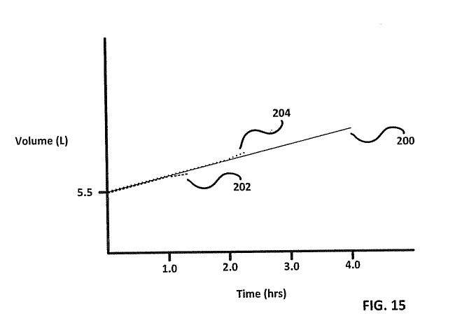

FIG. 15 demonstrates the target fluid removal profile line 200 (ultrafiltration profile) for a prescribed therapy and the consequences (lines 202 and 204) of blood extracorporeal circuit 72 occlusions. While in treatment steady state (following a prescribed ultrafiltration profile, e.g. line 200) each of the three peristaltic pumps (40, 42 and 98) are at a fixed rate or revolutions per minute (RPM). The susceptibility of peristaltic pumps (40, 42 and 98) to changes in inlet fluid pressure allow this system to use those abrupt, unexpected fluctuations (as a result of fluid line occlusions) in the therapeutic fluid side pressure and the subsequent effect on flow through the dialyzer 70 as a means to detecting fluid line occlusions. If an unexpected blood extracorporeal circuit occlusion occurs in the venous line 94 prior to the blood pump 98 the system will immediately detect a pressure drop at first pressure sensor 102 and in conjunction with this the actual fluid removal profile line 202 will immediately start to fall off of the target profile line 200. Line 202 demonstrates what that deviation from the target line 200 may look like should such an occlusion occur at approximately one hour into the blood therapy treatment. If an unexpected blood extracorporeal circuit occlusion occurs post-third peristaltic pump 98 but pre-dialyzer 70 the system will immediately detect a pressure spike at second pressure sensor 104 and in conjunction with this the actual fluid removal profile line 202 will immediately start to fall off of the target profile line 200. Line 202 shows what that deviation from the target line 200 may look like should such an occlusion occur at approximately one hour into the blood therapy treatment. If an unexpected blood extracorporeal circuit occlusion occurs in the arterial line 92, post-dialyzer 70, but pre-patient 125 the system will immediately detect a pressure spike at second pressure sensor 104 and in conjunction with this the actual fluid removal profile line 204 will immediately pull away from the target profile line 200. Line 204 shows what that deviation from the target line 200 may look like should such an occlusion occur at approximately two hours into the blood therapy treatment.

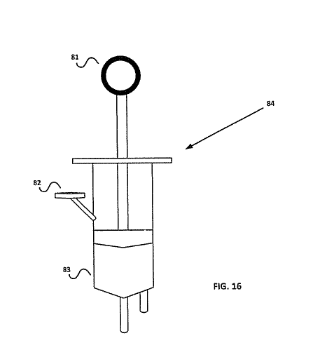

FIG. 16 depicts a syringe style blood air trap 84. As part of the blood extracorporeal circuit 72, the syringe style blood air trap 84 is integral in purging air both during the priming process and throughout the duration of the blood treatment session. In the single needle therapy configuration it acts to assist in the to-and-fro flow to the patient through the single fistula needle. When mounted to the system hardware (not shown), fluid sensors 88 and 86 (FIG. 4, FIG. 5, FIG. 6, FIG. 7, FIG. 8, FIG. 9, FIG. 10) are able to detect fluid in the syringe style blood air trap 84. Piston 81 is able to travel up and down syringe tube 83 adjusting the level. Piston 81 is also able to travel above the microbial membrane filter protected vent line 82 enabling the blood extracorporeal circuit 72 to be opened to atmosphere via a protective membrane.

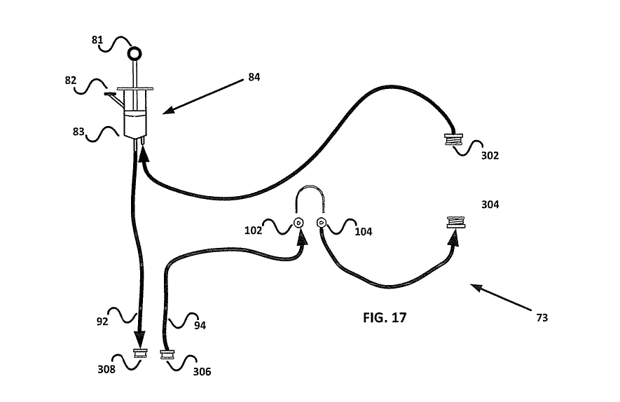

FIG. 17 displays the blood tubing set 73 prior to being integrated with the rest of the blood extracorporeal circuit 72. The venous blood line 94 is connected to the patient fistula via male luer connector 306. The venous blood line 94 feeds the third peristaltic pump 98 which is preceded by the first pressure sensor 102 and followed by the second pressure sensor 104. The venous blood line 94 then continues to the inlet of the dialyzer 70 where the connection is made via a standard male threaded dialyzer connector 304. The connection at the blood outlet end of the dialyzer 70 is also made via the same connector type 302. The venous blood line 94 then leads into the blood air trap 84 allowing the blood to enter into the syringe like cylinder 83. Blood arterial line 92 takes blood from the air trap 84 to the male luer connector 308 which is used to ultimately make the connection to the patient fistula.

In operation, a method for providing blood therapy includes the steps of providing a blood tubing set 73 and a therapeutic fluid source 38 supplying a therapeutic fluid 39. The blood tubing set 73 and the therapeutic fluid source 38 are separated by a dialyzer 70 having a transmembrane pressure. A first tube 44 is in fluid communication between the therapeutic fluid source 38 and the dialyzer 70. A second tube 46 is in fluid communication between the therapeutic fluid source 38 and the dialyzer 70. A first peristaltic pump 40 is in cooperative engagement with the first tube 44. A second peristaltic pump 42 in cooperative engagement with the second tube 46. A third peristaltic pump 98 is in cooperative engagement with the blood tubing set 73, the third peristaltic pump 73 having a first pressure sensor 102 in cooperative engagement with the inlet of the third peristaltic pump 98 and a second pressure sensor 104 in cooperative engagement with the outlet of the third peristaltic pump 98. A programmable pressure control scheme (not shown) having a feedback loop is cooperatively engaged with the first peristaltic pump, the second peristaltic pump, and the third peristaltic pump to monitor and control the transmembrane pressure without invasively contacting said therapeutic fluid.

### Blood Therapy Configuration

### Single Needle

In instances where it is desirable for the patient to sleep during the blood therapy session, Single Needle configuration as demonstrated in FIG. 10 is desired in order to mitigate the potential risk of venous needle (FIGS. 6, 7, 8 & 9) dislodgement. In conventional dialysis configuration venous needle dislodgement could result in patient exsanguination. It should become apparent that the same Blood Therapy States described above using the conventional dialysis configuration (dual needle FIGS. 6, 7, 8 & 9) can be utilized in the Single Needle (FIG. 10) configuration with only slight modifications.

In Single Needle configuration blood flow to-and-fro the patient can be augmented by use of the syringe-like air trap 84. By raising and lowering the piston 81 of the air trap 84, blood can be pulled-in-to and pushed-out-of the blood extracorporeal circuit 72. With the blood pump running continuously or even intermittently, the air trap piston 81 can be used to increase or decrease the blood volume in the blood extracorporeal circuit 72, thereby creating blood flow to-and-fro the patient.

Hence, while the invention has been described in connection with a preferred embodiment and alternative embodiments, it will be understood that it is not intended that the invention be limited to those embodiments. On the contrary, it is intended to cover all alternatives, modifications and equivalents as may be included within the spirit and scope of the invention as disclosed.

As to the manner of usage and operation of the instant invention, same should be apparent from the above disclosure, and accordingly no further discussion relevant to the manner of usage and operation of the instant invention shall be provided.

With respect to the above description then, it is to be realized that the optimum proportions for the elements of the invention, and variations in size, materials, shape, form, function and manner of operation, assembly and use, are deemed readily apparent and obvious to one skilled in the art, and all equivalent relationships described in the specification are intended to be encompassed by the present invention.

Therefore, the foregoing is considered illustrative of only the principles of the invention. Further, since numerous modifications and changes will readily occur to those skilled in the art, it is not desired to limit the invention to the exact method, construction and operation shown and described, and accordingly, all suitable modifications and equivalents may be resorted to, falling within the scope of the invention.

What is claimed is:

1. A system for preparation of therapeutic fluid comprising:
a first fluid container containing a first therapeutic fluid component, said first therapeutic fluid component having a first known mass;
a second fluid container containing a second therapeutic fluid component, said second therapeutic fluid component having a second known mass;
said first therapeutic fluid component and said second therapeutic fluid component disposed in fluid communication with each other;
a fluid mixing system provided in cooperative engagement with said first therapeutic fluid component and said second therapeutic fluid component;
a volume or weight monitoring system in cooperative engagement with said first therapeutic fluid container, said second therapeutic fluid container, and said fluid mixing system;
a weight control scheme cooperatively engaged with said volume or weight monitoring system and said fluid mixing and warming system and programmed to operate said fluid mixing system to facilitate mixing and warming of said first therapeutic fluid component and said second therapeutic fluid component to form a therapeutic fluid of target body temperature; and
at least one heating element 97 disposed to warm the therapeutic fluid.

2. The system for preparation of therapeutic fluid of claim 1, wherein said fluid mixing and warming system comprises:
a first peristaltic pump and a second peristaltic pump;
a filter bypass having a first end and a second end;
a thermally insulated fluid warming chamber containing a low power heater;
a first tube in fluid communication between said first fluid container and said first end of said filter bypass, said first peristaltic pump cooperatively engaging said first tube; and
a second tube in fluid communication between said second fluid container and said second end of said filter bypass, said second peristaltic pump cooperatively engaging said second tube, said first peristaltic pump and said second peristaltic pump being configured to pump said first therapeutic fluid component and said second therapeutic fluid component between said first fluid container and said second fluid container.

3. The system for preparation of therapeutic fluid of claim 1, wherein said volume or weight monitoring system comprises:
a first load cell, a second load cell, and a strain gauge, said first load cell, said second load cell, and said strain gauge being engaged by said weight control scheme.

4. The system for preparation of therapeutic fluid of claim 2, wherein said weight monitoring system comprises:
a first load cell, a second load cell, and a strain gauge, said first load cell, said second load cell, and said strain gauge being engaged by said weight control scheme.

5. The system for preparation of therapeutic fluid of claim 4, wherein said weight control scheme further comprises:
a feedback loop from said first load cell, said second load cell 34, and said strain gauge to said first peristaltic pump and said second peristaltic pump to facilitate mixing of the first therapeutic fluid component and the second therapeutic fluid component to form a therapeutic fluid.

6. A method of preparation of therapeutic fluid comprising the steps of:
providing a first fluid container containing a first therapeutic fluid component, said first therapeutic fluid component having a first known mass;
providing a second fluid container containing a second therapeutic fluid component, said second therapeutic fluid component having a second known mass;
disposing said first therapeutic fluid component and said second therapeutic fluid component in fluid communication with each other;
providing a fluid mixing system in cooperative engagement with said first therapeutic fluid component and said second therapeutic fluid component;
providing a weight monitoring system in cooperative engagement with said first therapeutic fluid container, said second therapeutic fluid container, and said fluid mixing system; and
programming a weight control scheme cooperatively engaged with said weight monitoring system and said fluid mixing system to operate said fluid mixing system to facilitate mixing of said first therapeutic fluid component and said second therapeutic fluid component to form a therapeutic fluid.

7. The system for preparation of therapeutic fluid of claim 3 further comprising keyed mechanisms provided in connection with said first load cell and said second load cell to allow connection only to corresponding said first fluid container and said second fluid container.

8. The system for preparation of therapeutic fluid of claim 4 further comprising keyed mechanisms provided in connection with said first load cell and said second load cell to allow connection only to corresponding said first fluid container and said second fluid container.

9. A blood therapy system comprising:
a blood compartment and a therapeutic fluid compartment, said blood compartment and said therapeutic fluid compartment separated by a blood filtering device having a transmembrane pressure;
a blood extracorporeal circuit in fluid engagement with said blood compartment;
a therapeutic fluid circuit in fluid engagement with said therapeutic fluid compartment;
a therapeutic fluid source in fluid communication with said therapeutic fluid circuit containing a therapeutic fluid having a third known mass;
a fluid mixing system provided in cooperative engagement with a therapeutic fluid source;
a volume or weight monitoring system in cooperative engagement with said therapeutic fluid source; and
a weight control scheme cooperatively engaged with said volume or weight monitoring system and said fluid mixing system and programmed to operate said fluid mixing system to facilitate the delivery and monitoring of said therapeutic fluid from said therapeutic fluid source, through said therapeutic fluid circuit, into said therapeutic fluid compartment, through said blood filtering device, and into said blood extracorporeal circuit, wherein said weight control scheme monitors and controls the transmembrane pressure without invasively contacting said therapeutic fluid.

10. The blood therapy system of claim 9, wherein said fluid mixing system comprises:
a first peristaltic pump and a second peristaltic pump;
a first tube in fluid communication between said therapeutic fluid source and said therapeutic fluid compartment, said first peristaltic pump cooperatively engaging said first tube;
a second tube in fluid communication between said therapeutic fluid source and said therapeutic fluid compartment, said second peristaltic pump cooperatively engaging said second tube;
said first peristaltic pump and said second peristaltic pump being configured to pump said therapeutic fluid between said therapeutic fluid source and said therapeutic fluid compartment.

11. The blood therapy system of claim 9, wherein said volume or weight monitoring system comprises one or more load cells engaging said therapeutic fluid source and said weight control scheme.

12. The blood therapy system of claim 10, wherein said volume or weight monitoring system comprises one or more load cells engaging said therapeutic fluid source and said weight control scheme.

13. The blood therapy system of claim 12, wherein said control weight scheme further comprises:
a feedback loop from said load cells to said first peristaltic pump and said second peristaltic pump to facilitate the delivery and monitoring of said therapeutic fluid from said therapeutic fluid source, through said therapeutic fluid circuit, into said therapeutic fluid compartment, through said blood filtering device, and into said blood extracorporeal circuit.

14. A method of providing blood therapy comprising the steps of:
providing a blood compartment and a therapeutic fluid compartment, said blood compartment and said therapeutic fluid compartment separated by a blood filtering device;
providing a blood extracorporeal circuit side of the system in fluid engagement with said blood compartment;
providing a therapeutic fluid circuit side of the system in fluid engagement with said therapeutic fluid compartment;
disposing a therapeutic fluid source in fluid communication with said therapeutic fluid circuit containing a therapeutic fluid having a third known mass;
providing a fluid mixing system in cooperative engagement with a therapeutic fluid source;
providing a volume or weight monitoring system in cooperative engagement with said therapeutic fluid source; and
programming a weight control scheme cooperatively engaged with said volume or weight monitoring system and said fluid mixing system to operate said fluid mixing system to facilitate the delivery and monitoring of said therapeutic fluid from said therapeutic fluid source, through said therapeutic fluid circuit, into said therapeutic fluid compartment, through said blood filtering device, and into said blood extracorporeal circuit, wherein said control weight scheme does not invasively contact said therapeutic fluid.

15. A blood therapy system comprising:
a blood compartment and a therapeutic fluid compartment, said blood compartment and said therapeutic fluid compartment separated by a blood filtering device having a transmembrane pressure;
a blood extracorporeal circuit in fluid engagement with said blood compartment;
a therapeutic fluid circuit in fluid engagement with said therapeutic fluid compartment;
a first fluid container containing a first therapeutic fluid component, said first therapeutic fluid component having a first known mass;
a second fluid container containing a second therapeutic fluid component, said second therapeutic fluid component having a second known mass;
said first therapeutic fluid component and said second therapeutic fluid component disposed in fluid communication with each other;
a fluid mixing system provided in cooperative engagement with said first therapeutic fluid component and said second therapeutic fluid component;
a volume or weight monitoring system in cooperative engagement with said first therapeutic fluid container, said second therapeutic fluid container, and said fluid mixing system;
a control weight scheme cooperatively engaged with said volume or weight monitoring system and said fluid mixing system and programmed to operate said fluid mixing system to facilitate mixing of said first therapeutic fluid component and said second therapeutic fluid component to form a therapeutic fluid; and
said control weight scheme cooperatively engaged with said weight monitoring system and said fluid mixing system and programmed to operate said fluid mixing system to facilitate delivery and monitoring of said therapeutic fluid from said first therapeutic fluid container and said second therapeutic fluid container, through said therapeutic fluid circuit, into said therapeutic fluid compartment, through said blood filtering device, and into said blood extracorporeal circuit, wherein said weight control scheme monitors and controls the transmembrane pressure without invasively contacting said therapeutic fluid.

16. The blood therapy system of claim 15, wherein said fluid mixing system comprises:
a first peristaltic pump and a second peristaltic pump;
a first tube in fluid communication between said first fluid container and said therapeutic fluid compartment, said first peristaltic pump cooperatively engaging said first tube;
a second tube in fluid communication between said second fluid container and said therapeutic fluid compartment, said second peristaltic pump cooperatively engaging said second tube;
said first peristaltic pump and said second peristaltic pump being configured to facilitate delivery and monitoring of said therapeutic fluid from said first therapeutic fluid container and said second therapeutic fluid container, through said therapeutic fluid circuit, into said therapeutic fluid compartment, through said blood filtering device, and into said blood extracorporeal circuit.

17. The blood therapy system of claim 15, wherein said weight monitoring system comprises a first load cell, a second load cell, and a strain gauge, said first load cell, said second load cell, and said strain gauge being engaged by said weight control scheme.

18. The blood therapy system of claim 16, wherein said weight monitoring system comprises a first load cell, a second load cell, and a strain gauge, said first load cell, said second load cell, and said strain gauge being engaged by said weight control scheme.

19. The blood therapy system of claim 18, wherein said weight control scheme further comprises:
a feedback loop from said first load cell, said second load cell, and said strain gauge to said first peristaltic pump and said second peristaltic pump to facilitate the delivery and monitoring of said therapeutic fluid from said first therapeutic fluid container and said second therapeutic fluid container, through said therapeutic fluid circuit, into said therapeutic fluid compartment, through said blood filtering device, and into said blood extracorporeal circuit.

20. A method of providing blood therapy comprising the steps of:
providing a blood compartment and a therapeutic fluid compartment; said blood compartment and said therapeutic fluid compartment separated by a blood filtering device having a transmembrane pressure;
providing a first fluid container containing a first therapeutic fluid component, said first therapeutic fluid component having a first known mass;
providing a second fluid container containing a second therapeutic fluid component, said second therapeutic fluid component having a second known mass;
disposing said first therapeutic fluid component and said second therapeutic fluid component in fluid communication with each other;
providing a fluid mixing system in cooperative engagement with said first therapeutic fluid component and said second therapeutic fluid component;
providing a weight monitoring system in cooperative engagement with said first therapeutic fluid container, said second therapeutic fluid container, and said fluid mixing system; and
programming a weight control scheme cooperatively engaged with said weight monitoring system and said fluid mixing system to operate said fluid mixing system to facilitate the delivery and monitoring of said therapeutic fluid from said first therapeutic fluid container and said second therapeutic fluid container, through said therapeutic fluid circuit, into said therapeutic fluid compartment, through said blood filtering device, and into said blood extracorporeal circuit, wherein said weight control scheme monitors and controls the transmembrane pressure without invasively contacting said therapeutic fluid.

21. The blood therapy system of claim 9, wherein said extracorporeal blood circuit further comprises:
a venous line and an arterial line removably connected to each other in fluid communication by a recirculation connector line for the purposes of priming the blood tubing set through said blood filtering device.

22. The blood therapy system of claim 10, wherein said extracorporeal blood circuit further comprises:
a venous line and an arterial line removably connected to each other in fluid communication by a recirculation connector line for the purposes of priming the blood tubing set through said blood filtering device.

23. The method of providing blood therapy of claim 14 further comprising the steps of:
removably connected a venous line and an arterial line to each other in fluid communication by a recirculation connector line for the purposes of priming the blood tubing set through said blood filtering device.

24. The blood therapy system of claim 15, wherein said extracorporeal blood circuit further comprises:
a venous line and an arterial line removably connected to each other in fluid communication by a recirculation connector line for the purposes of priming the blood tubing set through said blood filtering device.

25. The blood therapy system of claim 16, wherein said extracorporeal blood circuit further comprises:
a venous line and an arterial line removably connected to each other in fluid communication by a recirculation connector line for the purposes of priming the blood tubing set through said blood filtering device.

26. The method of providing blood therapy of claim 20 further comprising the steps of:
removably connected a venous line and an arterial line to each other in fluid communication by a recirculation connector line for the purposes of priming the blood tubing set through said blood filtering device.

27. The blood therapy system of claim 9, wherein said system further comprises:
a venous line and an arterial line removably connected via removable tubes to said therapeutic fluid source container for the purposes of priming the blood tubing set.

28. The blood therapy system of claim 10, wherein said system further comprises:
a venous line and an arterial line removably connected via removable tubes to said therapeutic fluid source container for the purposes of priming the blood tubing set.

29. The method of providing blood therapy of claim 14 further comprising the steps of:
removably connected a venous line and an arterial line via removable tubes to said therapeutic fluid source container for the purposes of priming the blood tubing set.

30. The blood therapy system of claim 15, wherein said system further comprises:
a venous line and an arterial line removably connected via removable tubes to said first therapeutic fluid container and said second therapeutic fluid container for the purposes of priming the blood tubing set.

31. The blood therapy system of claim 16, wherein said system further comprises:
a venous line and an arterial line removably connected via removable tubes to said first therapeutic fluid container and said second therapeutic fluid container for the purposes of priming the blood tubing set.

32. The method of providing blood therapy of claim 20 further comprising the steps of:
removably connected a venous line and an arterial line via removable tubes to said first therapeutic fluid container and said second therapeutic fluid container for the purposes of priming the blood tubing set.

33. The method of providing blood therapy of claim 14 further comprising the steps of:
programming said weight control scheme cooperatively engaged with said weight monitoring system and said fluid mixing system to operate said fluid mixing system to facilitate the delivery and monitoring of said therapeutic fluid from said first therapeutic fluid container and said second therapeutic fluid container, through said therapeutic fluid circuit, into said therapeutic fluid compartment, through said blood filtering device, and into said blood extracorporeal circuit for the purposes of ultrafiltration.

34. The method of providing blood therapy of claim 20 further comprising the steps of:
programming said weight control scheme cooperatively engaged with said weight monitoring system and said fluid mixing system to operate said fluid mixing system to facilitate the delivery and monitoring of said therapeutic fluid from said first therapeutic fluid container and said second therapeutic fluid container, through said therapeutic fluid circuit, into said therapeutic fluid compartment, through said blood filtering device, and into said blood extracorporeal circuit for the purposes of ultrafiltration.

35. The method of providing blood therapy of claim 14 further comprising the steps of:
programming said weight control scheme cooperatively engaged with said weight monitoring system and said fluid mixing system to operate said fluid mixing system to facilitate the delivery and monitoring of said therapeutic fluid from said first therapeutic fluid container and said second therapeutic fluid container, through said therapeutic fluid circuit, into said therapeutic fluid compartment, through said blood filtering device, and into said blood extracorporeal circuit for the purposes of ultrafiltration.

36. The method of providing blood therapy of claim 20 further comprising the steps of:
programming said weight control scheme cooperatively engaged with said weight monitoring system and said fluid mixing system to operate said fluid mixing system to facilitate the delivery and monitoring of said therapeutic fluid from said first therapeutic fluid container and said second therapeutic fluid container, through said therapeutic fluid circuit, into said therapeutic fluid compartment, through said blood filtering device, and into said blood extracorporeal for the purposes of ultrafiltration.

37. A method of providing blood therapy comprising the steps of:
providing a blood tubing set and a therapeutic fluid source supplying a therapeutic fluid, said blood tubing set and said therapeutic fluid source separated by a blood filtering device having a transmembrane pressure;
providing a first tube in fluid communication between said therapeutic fluid source and said blood filtering device;
providing a second tube in fluid communication between said therapeutic fluid source and said blood filtering device;
providing a first peristaltic pump in cooperative engagement with said first tube;
providing a second peristaltic pump in cooperative engagement with said second tube;
providing a third peristaltic pump in cooperative engagement with said blood tubing set, said third peristaltic pump having a first pressure sensor in cooperative engagement with the inlet of said third peristaltic pump and a second pressure sensor in cooperative engagement with the outlet of said third peristaltic pump;
programming a pressure control scheme cooperatively engaged with said first peristaltic pump, said second peristaltic pump, and said third peristaltic pump to monitor and control the transmembrane pressure without invasively contacting said therapeutic fluid.
